(19) United States
(12) Patent Application Publication
(10) Pub. No.: US 2007/0168010 A1
(43) Pub. Date: Jul. 19, 2007

(54) Hinged Stent

(75) Inventor: Justin Goshgarian, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/275,607

(22) Filed: Jan. 19, 2006

(51) Int. Cl. A61F 2/06

(52) U.S. Cl. 623/1.11

(57) ABSTRACT

A stent delivery system is provided that comprises an inner member and an expandable balloon mounted on the inner member. A stent, which is mounted around at least a portion of the expandable balloon, comprises a plurality of alternating, hingedly-coupled crown sections and strut sections. Each adjacent crown section and strut section is coupled together via a hinge comprising a region having a thickness substantially less than that of the adjacent crown section and strut section.

### TECHNICAL FIELD

This invention relates generally to an implantable stent apparatus and, more particularly, to a hinged stent having improved radial strength when in an expanded state.

### BACKGROUND OF THE INVENTION

Cardiovascular disease is a leading cause of death. Consequently, the medical community has devised various methods and devices for the treatment of coronary heart disease. One such treatment utilized in cases involving atherosclerosis and/or other forms of coronary narrowing is referred to as percutaneous transluminal coronary angioplasty, sometimes simply referred to as angioplasty or PTCA. The objective of this technique is to radially enlarge the lumen of the impacted vessel by positioning an expandable balloon proximate a targeted lesion (e.g., through the narrowed lumen of the coronary artery) and inflating the balloon. Inflation of the balloon enlarges the lumen of the vessel by flattening soft or fatty plaque deposits, breaking up hardened deposits, and stretching the vessel's walls.

In a typical PTCA procedure, a passageway into the patient's cardiovascular system is created through a relatively large vessel, such as the femoral artery in the groin area or the brachial artery in the arm. A guide catheter is inserted into the passageway and guided to the ostium of the vessel to be treated and a flexible guide wire is introduced into the guide catheter and advanced to the target lesion. A balloon or dilatation catheter is then advanced over the guide wire until the dilatation balloon is properly positioned across the target lesion. Radiopaque markers, which may be fluoroscopically viewed, are disposed proximate the balloon portion of the dilatation catheter and assist in the positioning of the balloon across the lesion. After proper positioning, the balloon is inflated (e.g., preferably with a contrast material to enhance fluoroscopic viewing during the treatment) thereby enlarging the vessel's lumen. Treatment may require that the balloon be alternately inflated and deflated until satisfactory enlargement has been achieved. The balloon is then deflated to a small profile so that the dilatation catheter may be withdrawn from the patient's vasculature and blood flow resumed through the dilated vessel.

Unfortunately, after angioplasty procedures of the type described above, there may occur a restenosis of the treated vessel (i.e., a renarrowing of the vessel), which may significantly diminish any positive results of the angioplasty procedure. In the past, restenosis frequently necessitated repeat PTCA and occasionally open-heart surgery. To prevent restenosis and strengthen the target area, mechanical endoprosthetic devices have been developed. Such devices, which are generally referred to as stents, physically maintain the expanded diameter of a treated vessel after completion of the angioplasty procedure. Typically, a stent is mounted in a compressed state around a deflated balloon, and the balloon/stent assembly is maneuvered through a patient's vasculature to the site of the target lesion. The balloon is then inflated thereby causing the stent to expand to a larger diameter suitable for implantation in the vasculature. The stent effectively overcomes the natural tendency of the vessel walls to renarrow by providing a scaffolding-like support.

Many types of stents have been proposed and utilized. One known stent comprises a stainless steel wire braid that is bent to form a generally cylindrical tube, which is positioned on a delivery device and deployed in the manner described above. Another known stent, which is commonly referred to as a Palmaz stent, utilizes a stainless steel cylinder having a number of slits in its circumference resulting in a mesh when expanded. A more detailed discussion of the Palmaz stent may be found in U.S. Pat. No. 4,733,665, the teachings of which are hereby incorporated by reference.

Unfortunately, conventional stents including those of the type described above are known to suffer from elastic recoil; i.e., collapse under the inward radial pressure exerted thereon by vessel walls. If the collapse is partial, the deployed stent will not be uniformly dilated and will thus be structurally weakened. If the collapse is total, the deployed stent will be rendered ineffective and may become an obtrusion. In view of this, it should be appreciated that it would be desirable to provide a stent with a relatively high radial strength (i.e., a greater load bearing capacity when in an expanded state) that is less likely to collapse when deployed within a patient's vasculature. Furthermore, other desirable features and characteristics of the present invention will become apparent from the subsequent detailed description of the invention and the appended claims, taken in conjunction with the accompanying drawings and this background of the invention.

### SUMMARY OF THE INVENTION

A stent delivery system is provided that comprises an inner member and an expandable balloon mounted on the inner member. A stent, which is mounted around at least a portion of the expandable balloon, comprises a plurality of alternating, hingedly-coupled crown sections and strut sections. Each adjacent crown section and strut section is coupled together via a hinge comprising a region having a thickness substantially less than that of the adjacent crown section and strut section.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and:

FIG. 1 is a side view, partially in cross-section, of a conventional balloon/stent assembly;

FIG. 2 is a side view of a section of the stent illustrated in FIG. 1 in an unfurled state;

FIGS. 3 and 4 are side views of a stent section unit in compressed and expanded (deployed) states, respectively;

FIG. 5 is a side view of a stent section in accordance with a first embodiment of the present invention;

FIG. 6 and 7 are side views of a stent section unit in compressed and expanded (deployed) states, respectively, in accordance with the present invention;

FIG. 8 is a side view of the stent section unit illustrated in FIGS. 6 and 7 having rounded edges; and

FIG. 9 is a side view of a stent section unit in accordance with a second embodiment of the present invention.

### DETAILED DESCRIPTION OF THE EXEMPLARY EMBODIMENT

The following description is exemplary in nature and is not intended to limit the scope, applicability, or configuration of the invention in any way. Rather, the following description provides a convenient illustration for implementing an exemplary embodiment of the invention. Various changes to the described embodiment may be made in the function and arrangement of the elements described herein without departing from the scope of the invention.

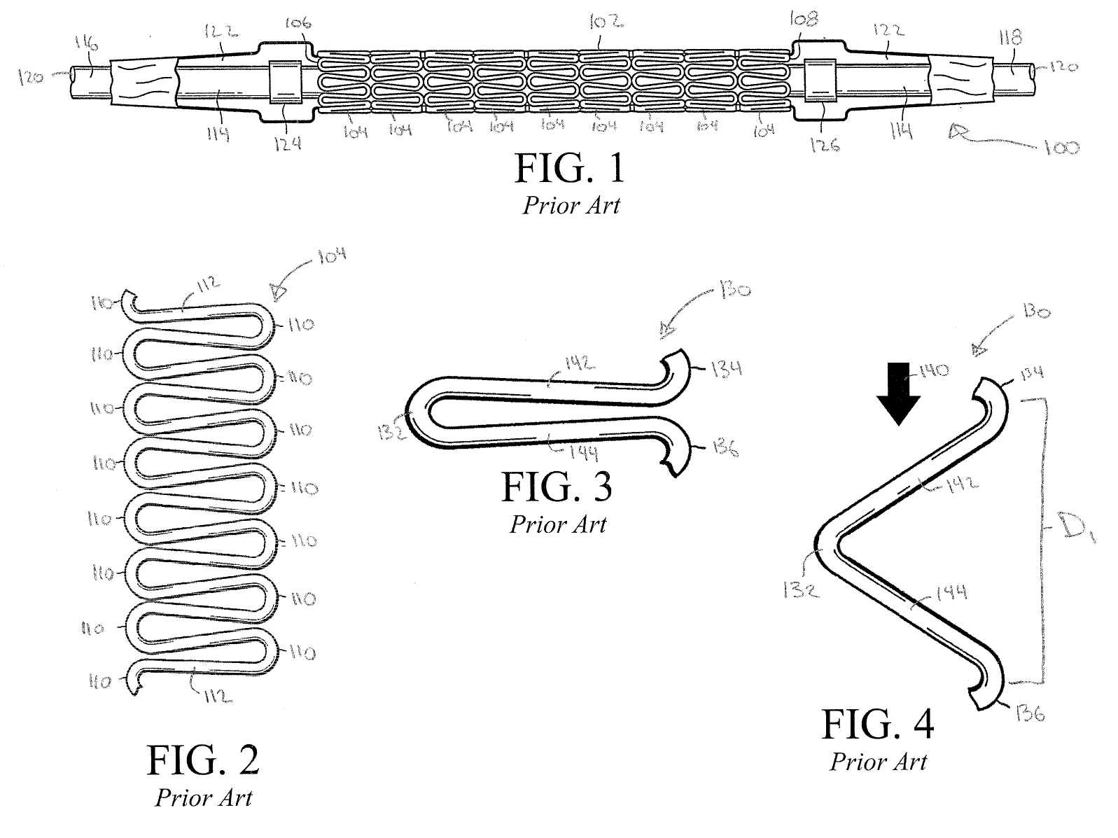

FIG. 1 is a side view, partially in cross-section, of a balloon/stent assembly 100 that is configured to support and deliver an endovascular support device such as a stent 102 to a target area inside a patient's body (e.g., an artery affected by atherosclerosis). Stent 102 comprises at least one stent section 104 (nine such sections are shown in FIG. 1), which are coupled together in the well-known manner (e.g., welding) to create a generally tubular mesh body having a proximal end 106 and a distal end 108. Stent 102 may be constructed of any implantable material having good mechanical strength, such as stainless steel, tantalum, super-elastic nickel-titanium alloys, or high-strength thermoplastic polymers. The cross-sectional shape of stent 102 may be circular, ellipsoidal, rectangular, hexagonal, square, or any other desired shape, although a circular or ellipsoidal cross-section is preferable. The length and width of stent 102 are generally dictated by the size of the vessel to be treated; stent 102 must be of sufficient length to extend across a significant portion of the target area while maintaining its axial orientation without shifting under the hydraulics of blood flow. At the same time, stent 102 should not be unnecessarily long so as to result in the introduction of a large amount of material into the vessel. If desired, an outer portion of stent 102 may be plated with platinum or other implantable radiopaque substance to provide fluoroscopic visibility.

FIG. 2 illustrates a single stent section 104 in an unfurled state. Stent section 104 comprises a plurality of axially bends 110 (commonly referred to as crowns) that are interconnected by a plurality of elongated segments 112 (commonly referred to as struts) to form a serpentine-like mesh, which may expand (or, more accurately, be expanded) along the circumference of stent 102. Stent section 104 may be produced via any one of a number of known methods. For example, section 104 may be produced from a machined wire ring or torroid (e.g., machined from stainless steel bar stock), which is then bent or formed into the desired shape. Alternatively, section 104 may be produced by cutting a tubular ring made of an implantable metal with, for example, a laser. After manufacture, stent section 104 is coupled to similar stent sections to form stent 102. More specifically, each of crowns 110 is coupled (e.g., welded) to a different one of crowns 110 on an adjacent stent section 104 (except at the stent's proximal and distal ends) as shown in FIG. 1.

Referring still to FIG. 1, stent 102 is provided with first and second openings through proximal end 106 and distal end 108, respectively. Stent 102 is mounted along an inner member or tubing 114, which includes a proximal end 116, a distal end 118, and a wire lumen 120. An expandable balloon 122 is disposed around a portion of tubing 114 and passes through stent 102 such that the inflation of balloon 122 results in the radial expansion of stent 102. Generally, balloon 122 is made of a pliable material such as polyethylene, polyethylene terathalate, PEBAX (polyamide block copolymers and polyester block copolymers), polyvinyl chloride, polyolefin, nylon or the like. The length and the diameter of the balloon may be selected to accommodate the particular configuration of the stent to be deployed. The shape of balloon 122 is set in the following manner. An inner sheath (not shown) is placed over each end of balloon 122, and an exterior sheath (also not shown) is placed over the ends of the interior sheath so as to cover stent 102 and overlap with the interior sheath. Assembly 100 is then pressurized by introducing air or an inert gas (e.g., nitrogen) through lumen 120 and into the interior of balloon 122, which expands within the sheaths. Next, assembly 100 is exposed to an elevated temperature while the pressurization of balloon 122 is maintained at desired pressure. Lastly, balloon/stent assembly 100 is allowed to cool within the sheaths thereby setting the shape of balloon 122. In addition, in an alternative process, the heating of the stent assembly is limited to the balloon areas adjacent the stent ends to set balloon retainers or pillows. This process is described in detail in U.S. Pat. No. 5,836,965 entitled “Stent Delivery and Deployment Method” issued Nov. 17, 1998, the teachings of which are hereby incorporated by reference. To complete production of assembly 100, the sheaths are removed and stent 102 is compressed upon the outside of balloon 122.

Tubing 112 is configured to receive a conventional guide wire (now shown) at proximal end 116. The guide wire travels through wire lumen 120 to provide rigidity to tubing 114 and enable balloon/stent assembly 100 to be guided to and positioned within the targeted vessel. First and second radiopaque marker bands 124 and 126 are disposed around tubing 114 near the proximal and distal ends of stent 100, respectively. Marker bands 124 and 126 provide visibility during fluoroscopy to facilitate the proper positioning of balloon/stent assembly 100 across the lesion. When assembly 100 is properly positioned, a pressurized gas is introduced into lumen 114 causing the inflation of balloon 116 and the consequent expansion of stent 102. The amount of inflation and, thus the degree to which stent 102 is expanded, may be varied as required by the characteristics of the lesion. After stent 102 is satisfactorily deployed, balloon 116 is deflated and assembly 100 (minus stent 102) is withdrawn from the patient's vasculature.

For ease of understanding, stent section 104 may be thought of as comprising a plurality of repeating units 130. FIGS. 3 and 4 show one such unit 130 in compressed and expanded states, respectively. As can be seen, stent section unit 130 comprises one full crown 132 and two half crowns 134 and 136, which are coupled to crown 132 by way of first and second struts 142 and 144, respectively. In the compressed state shown in FIG. 3 (e.g., when stent 102 is mounted on assembly 100), a relatively small distance separates crowns 134 and 136 (in fact, crowns 134 and 136 may abut) and the longitudinal axes of struts 142 and 144 are substantially parallel. In contrast, in the expanded state shown in FIG. 4 (e.g., when stent 102 has been deployed in a patient's vasculature), a relatively large distance separates crowns 134 and 136 (distance D₁ in FIG. 4) and the longitudinal axes of struts 142 and 144 form a relatively large angle.

As described above, conventional stents such as stent 102 are known to suffer from elastic recoil, which occurs when a deployed stent collapses under the inward radial pressure exerted thereon by a vessel's walls. This inward radial pressure is applied to the stent circumferentially and may thus be thought of as a compression force that urges each stent section (and, therefore, each stent section unit) toward its compressed position. In the case of stent section unit 130 illustrated in FIG. 4, this compression force is represented by arrow 140. As will be appreciated by those adept in the art, the more vertical the struts relative to this compression force, the less load that will be applied thereto and the greater the overall radial strength of the stent. Unfortunately, stent 102 and other such prior art stents are incapable of an achieving optimal strut disposition. The present invention overcomes this drawback by providing a stent that achieves a more vertical strut disposition in its expanded (i.e., deployed) state and, consequently, a greater overall radial strength.

FIG. 5 illustrates a single stent section 200 in an unfurled state. Stent section 200 may be joined to other similar stent sections as is well-known to form a stent in accordance with a first embodiment of the present invention, which may then be deployed on a balloon/stent assembly (e.g., assembly 100) in the manner described above. As was the case with stent section 104 described above in conjunction with FIGS. 1-4, stent section 200 comprises a plurality of axially bends (i.e., crowns) 202 that are interconnected by a plurality of elongated segments (i.e., struts) 204 to form an expandable, serpentine-like mesh. Unlike stent section 104, however, stent section 200 further comprises a plurality of hinges 206; i.e., areas of reduced thickness relative to crowns 202 and/or struts 204 along axes substantially orthogonal to the longitudinal axis of a stent employing stent section 200. Preferably, hinges 206 each comprise a region having a thickness of approximately 50 to 75 less than that of crowns 2020 and/or struts 204. As described below, hinges 206 facilitate the bending of struts 204 relative to crowns 202 and thereby permit stent section 200 to achieve a more vertical strut disposition when expanded.

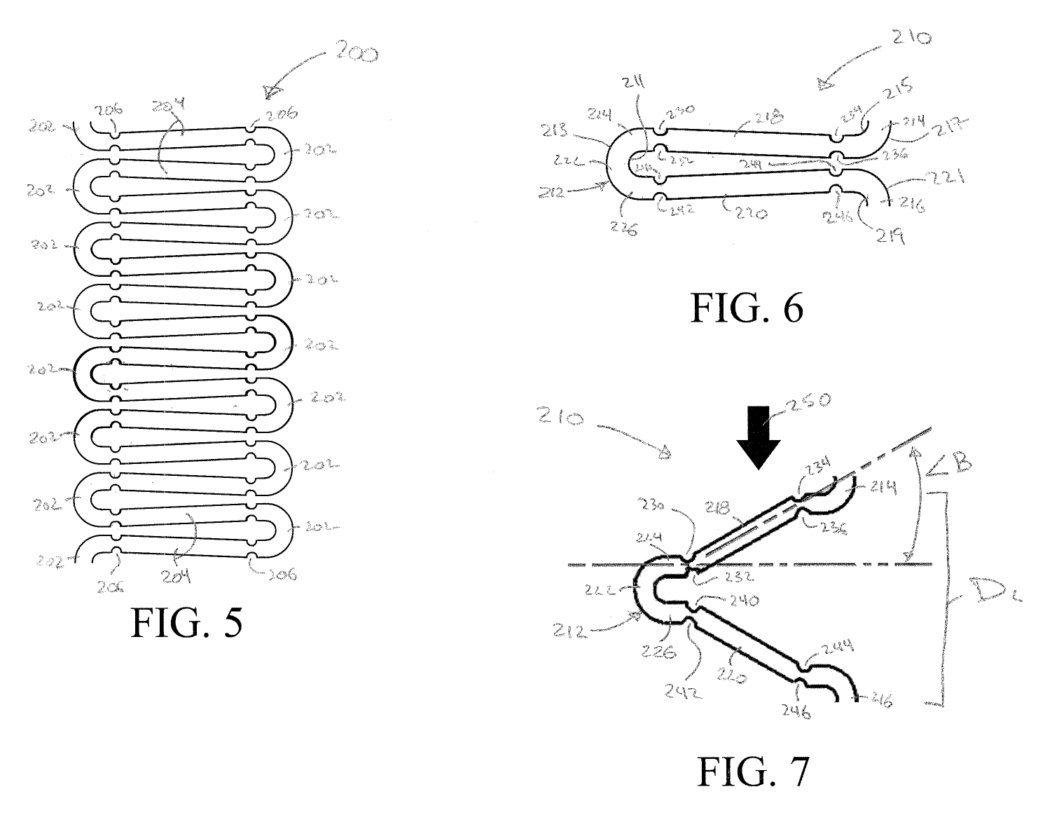

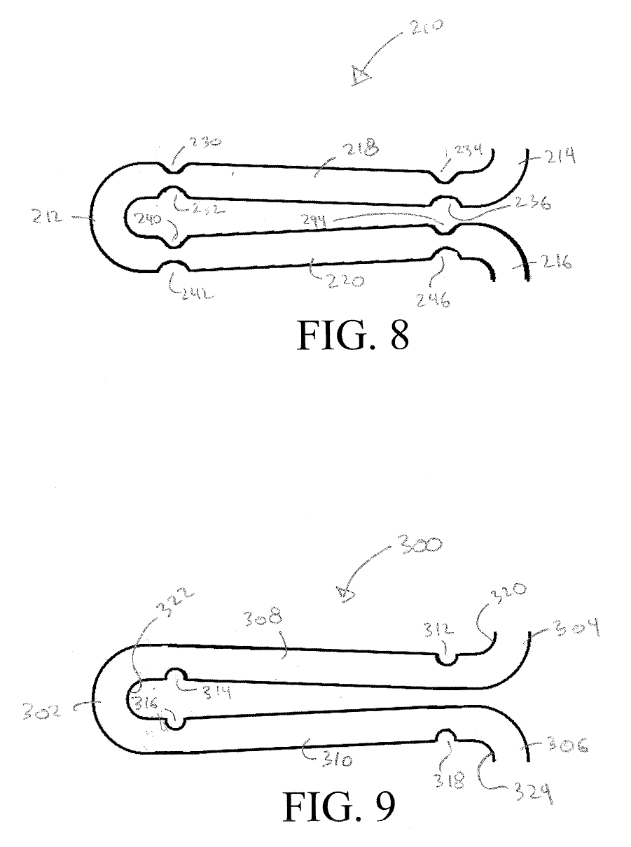

Again, for ease of understanding, stent section 200 may be conceptually divided into a plurality of repeating units. For example, stent section 200 may be thought of as comprising a plurality of J-shaped units each having one strut and one crown. Alternatively, stent section 200 may be thought of as comprising a plurality of U-shaped units 210, one of which is shown in FIGS. 6 and 7 in compressed and expanded states, respectively. Stent section unit 210 comprises one complete crown 212 and two partial crowns 214 and 216, which are each coupled to crown 212 by way of struts 218 and 220, respectively. Crown 212 has an apex 222 and first and second legs 224 and 226, which are coupled to struts 218 and 220, respectively. Preferably, apex 222 and legs 224 and 226 cooperate to provide a substantially U-shaped crown 212. Importantly, stent section unit 210 is provided with eight hinges (i.e., hinges 230, 232, 234, 236, 240, 242, 244, and 246), each of which is disposed between a crown and a strut. In particular, hinges 230 and 232 are disposed between crown 212 and strut 218, hinges 234 and 236 are disposed between crown 214 and strut 218, hinges 240 and 242 are disposed between crown 212 and strut 220, and hinges 244 and 246 are disposed between crown 216 and strut 220.

Hinges 230, 232, 234, 236, 240, 242, 244, and 246 each comprise an area of reduced thickness configured to facilitate the bending of struts 218 and 220 relative to crowns 212, 214, and 216. For each of the hinges, the area of reduced thickness is taken along an axis substantially orthogonal to the longitudinal axis of a stent employing one or more stent sections 200. As can be seen in FIG. 6, the hinges of stent section unit 210 are disposed as follows. Hinges 232 and 240 are disposed proximate an inner surface 211 of crown 212 along an inner periphery thereof, while hinges 230 and 242 are disposed proximate an outer surface 213 of crown 212 along an outer periphery thereof. Hinge 234 is disposed proximate an inner surface 215 of crown 214, and hinge 236 is disposed proximate an outer surface 217 of crown 214. Finally, hinge 246 is disposed proximate an inner surface 219 of crown 216, and hinge 244 is disposed proximate an outer surface 221 of crown 216.

In the compressed state (FIG. 6), crowns 214 and 216 are proximate each other and struts 218 and 220 are substantially parallel. In the expanded state shown in FIG. 7, however, crowns 134 and 136 have moved apart by a distance D₂ and struts 218 and 220 form a relatively large angle. Comparing FIG. 4 to FIG. 7, the relative spatial displacement of crowns 212, 214, and 216 of stent section unit 210 may be similar to the displacement of the crowns of stent section unit 130 when expanded (e.g., distance D₂ may be similar or equivalent to distance D₁); however, the vertical orientation of the struts 218 and 220 of unit 210 differs from those of unit 130. As can be seen in FIG. 7, hinges 230 and 232 permit strut 218 to bend relative to leg 224 of crown 212. This may be most easily appreciated in FIG. 7 by reference to angle B, which is formed between the longitudinal axes of strut 218 and leg 224. Similarly, hinges 234 and 236 facilitate the bending of strut 218 relative to crown 214, and hinges 240 and 242 and hinges 244 and 246 facilitate the bending of strut 220 relative to crown 212 and crown 216, respectively. It should thus be appreciated that the hinges provided between the crowns and the struts of unit 210 allow stent section unit 210, and consequently stent section 200, to achieve a more vertical strut disposition relative to the compression force represented in FIG. 7 by arrow 250. For the reasons described above, this allows stent section 200, and a stent employing one or more of sections 200, to achieve an improved radial strength relative to conventional stents.

Hinges, such as those described above, may be formed at various locations along a stent section in a number of ways. For example, the hinges may be notched into the stent section utilizing, for example, conventional laser-cutting equipment. This method may be particularly convenient if the stent section is produced by laser-cutting a tubular metal ring in the manner described above. Alternatively, the hinges may be created by a conventional swaging process. This method may be preferable if the stent section is produced by bending a machined wire as was also described above. It should be noted that, if a swaging method is utilized to create one or more of the hinges, material will not be removed from the stent section as it is during laser-cutting. Thus, if a swaging process is utilized, the outer diameter of the hinges may be equal to, or perhaps larger than, the outer diameter of the surrounding stent section; however, the stent section will have an area of reduced thickness along axes substantially orthogonal to the longitudinal axis of a stent employing one or more stent sections 200.

The hinges utilized in the inventive stent may have a variety of geometric profiles. For example, the hinges may have a cross-sectional profile that is substantially semi-circular, as described did the hinges described above in conjunction with stent section 200 (FIG. 5). If the geometry of the hinges comprises one or more edges, it may be desirable to smooth or round the hinge edges. As will be appreciated by one skilled in the art, this may be accomplished through the utilization of known polishing techniques (e.g., mechanical polishing, electrochemical polishing, etc.). FIG. 8 illustrates unit 210 (FIGS. 6 and 7) after undergoing such a polishing treatment. Comparing FIG. 8 to FIGS. 6 and 7, it can be seen that the edges of hinges 230, 232, 234, 236, 240, 242, 244, and 246 have each been substantially rounded.

FIG. 9 illustrates a stent section unit 300 that may be joined to similar units to form a stent section in accordance with another embodiment of the present invention. Stent section unit 300 comprises a complete crown 302 and two partial crowns 304 and 306, which are coupled to crown 302 by way of struts 308 and 310, respectively. Unit 300 is similar to unit 210 described above in conjunction with FIGS. 6 and 7; however, unlike unit 210, which comprised eight hinges, unit 300 comprises only four such hinges (i.e., hinges 312, 314, 316, and 318), which are disposed as follows. Hinge 312 is disposed between strut 308 and crown 304 proximate an inner peripheral surface 320 of crown 304. Hinge 314 is disposed between strut 308 and crown 302 proximate an inner peripheral surface 322 of crown 302, while hinge 316 is disposed between strut 310 and crown 302 proximate inner peripheral surface 322 of crown 304. Finally, hinge 318 is disposed between strut 310 and crown 306 proximate an inner peripheral surface 324 of crown 306. As previously alluded, hinges 314 and 316 facilitate the bending of struts 308 and 310, respectively, relative to crown 302; hinge 312 facilitates the bending of strut 308 relative to crown 304; and hinge 318 facilitates the bending of strut 310 relative to crown 306. It should thus be appreciated that, collectively, hinges 312, 314, 316, and 318 allow stent section unit 300 to achieve a more vertical strut disposition when in an expanded (deployed) state and, consequently, an improved radial strength.

In view of the foregoing specification, it should be appreciated that a stent having an improved radial strength relative to conventional stents has been provided, which is less likely to collapse when deployed within a patient's vasculature. Though the invention has been described with reference to a specific embodiment, it should be appreciated that various modifications and changes can be made without departing from the scope of the invention as set forth in the appended claims. Accordingly, the specification and figures should be regarded as illustrative rather than restrictive, and all such modifications are intended to be included within the scope of the present invention.

What is claimed is:

1. A stent delivery system, comprising:
an inner member;
an expandable balloon mounted on said inner member; and
a stent mounted around at least a portion of said expandable balloon, said stent comprising a plurality of alternating, hingedly-coupled crown sections and strut sections.

2. A stent delivery system according to claim 1 wherein each adjacent crown section and strut section is coupled together via a hinge comprising a region having a thickness substantially less than that of said adjacent crown section and strut section.

3. A stent delivery system according to claim 2 wherein said region of reduced thickness has a thickness approximately 50 to 75 percent less the thickness of said crown sections.

4. A stent delivery system according to claim 2 wherein each of said crown sections comprise:
a curved portion having a first end and a second end;
a first leg coupled to said curved portion at the first end thereof; and
a second leg coupled to said curved portion at the second end thereof.

5. A stent delivery system according to claim 4 wherein each of said crown sections has an inner peripheral surface, and wherein said region of reduced thickness resides proximate said inner peripheral surface between one of said strut sections and one of said first legs.

6. A stent delivery system according to claim 5 wherein additional regions of reduced thickness each reside proximate said inner peripheral surface between one of said strut sections and one of said second legs.

7. A stent delivery system according to claim 4 wherein each of said crown sections has an outer peripheral surface, and wherein said region of reduced thickness resides proximate said outer peripheral surface between one of said strut sections and one of said first legs.

8. A stent delivery system according to claim 7 wherein additional regions of reduced thickness each reside proximate said outer peripheral surface between one of said strut sections and one of said second legs.

9. A stent delivery system according to claim 4 wherein said crown sections are substantially U-shaped.

10. A stent delivery system according to claim 2 wherein said region of reduced thickness has substantially rounded edges.

11. A stent delivery system, comprising:
a tubing;
an expandable balloon mounted on said tubing; and
a stent mounted around at least a portion of said expandable balloon, said stent including at least one stent section comprising:
a plurality of successive crown sections each having first and second leg members;
a plurality of elongated strut sections each coupled between a first leg of a first one of said plurality of crown sections and a second leg of a second one of said plurality of said crown sections;
a first plurality of hinge regions each disposed between one of said strut sections and one of said first legs; and
a second plurality of hinge regions each disposed between one of said strut sections and one of said second legs.

12. A stent delivery system according to claim 11 wherein each of said plurality of crowns sections has an inner peripheral surface, and wherein each of said first plurality of hinge regions and each of said second plurality of hinge regions is disposed proximate said inner peripheral surface.

13. A stent delivery system according to claim 12 further comprising:
a third plurality of hinge regions each disposed between one of said strut sections and one of said first legs; and
a fourth plurality of hinge regions each disposed between one of said strut sections and one of said second legs.

14. A stent delivery system according to claim 10 wherein each of said crown sections is substantially U-shaped.

15. A stent delivery system according to claim 10 wherein each of said first plurality of hinge regions and each of said second plurality of hinge regions comprises a region of reduced thickness along an axis substantially orthogonal to the longitudinal axis of the stent.

16. An endovascular support device for mounting on a balloon catheter and configured to be expandably deployed in a patient's vasculature, the device comprising a plurality of J-shaped stent section units successively coupled together to form a serpentine-like mesh, each J-shaped section comprising:
a crown having a first leg member, a second leg member, and a curved portion; and
an elongated strut having a first end coupled to said first leg member and separated therefrom by a first region having a thickness substantially less than that of said first leg member proximate said first end to facilitate bending said strut relative to said crown when the support device is expanded.

17. A endovascular support device according to claim 16 wherein said elongated strut includes a second end and a second region of reduced thickness proximate said second end.

18. A method for producing a stent configured for radial expansion, comprising:
forming a generally tubular stent body comprising a plurality of alternatively coupled crown sections and strut sections; and
reducing the thickness of the stent body proximate selected crown/strut junctions to facilitate the bending of said strut sections relative to said crown sections.

19. A method according to claim 18 wherein each of the crown sections includes an inner peripheral surface and an outer peripheral surface, and wherein the step of reducing the thickness of the stent body comprises reducing the thickness proximate the inner peripheral surface and reducing the thickness proximate the outer peripheral surface.

20. A method according to claim 18 further comprising polishing the stent body to smooth edges produced by the step of reducing the thickness.
